(19) United States
(12) Patent Application Publication
(10) Pub. No.: US 2019/0099220 A1
(43) Pub. Date: Apr. 4, 2019

(54) A Transparent Electrode

(71) Applicant: Syneron Medical LTD., Yokneam lllit (IL)

(72) Inventors: Shmulik Eisenmann, Pardes Chana-Karkur (IL); Ital Kadosh, Jerusalem (IL); Kevin Schomacker, Maynard, MA (US)

(21) Appl. No.: 16/085,604

(22) PCT Filed: Apr. 6, 2017
(86) PCT No.: PCT/IL17/50415

§ 371 Date: Sep. 17, 2018

Related U.S. Application Data
(60) Provisional application No. 62/331,464, filed on May 4, 2016.

(51) Int. Cl. A61B 18/20; A61B 18/14

(52) U.S. Cl. CPC A61B 18/203; A61B 2018/00452; A61B 18/14

(57) ABSTRACT

An applicator for cosmetic treatment of skin including one or more sources of light energy and one or more transparent electrode assemblies positioned between the source of light energy and skin to be treated. The transparent electrode assembly supports transmission of light energy from the light source to one or more segments of skin located directly under and in contact with the electrode assembly. The electrode assembly can also include transparent RF electrodes that measure skin impedance (temperature).

### FIELD OF TECHNOLOGY

The apparatus and method are related to the field of cosmetic skin treatment and particularly to cosmetic skin treatment with electromagnetic energy.

### BACKGROUND

Cosmetic treatment of skin employing electromagnetic energy to heat segments of skin is well known. Such treatments commonly employ electromagnetic energy such as RF energy, coherent or incoherent light energy as well as other types of energy such as ultrasound energy to heat the skin.

Cosmetic skin treatment employing RF energy alone employing RF electrodes can produce non-uniform distribution of heat between the electrodes and “hot spots” resulting from the RF electrode geometry and configuration, commonly developing, for example, at edges of electrodes in contact with the skin and damaging of the skin. One attempt to limit the development of “hot spots” is use of a fractional or matrix array of electrodes as disclosed for example, in U.S. Pat. No. 8,357,150 to the same assignee.

It has also been found that for some cosmetic or other treatments, the combination of RF energy and light energy, employed concurrently, can be more efficient and hence advantageous in some cases over treatment with each type of energy alone (i.e., RF energy or light energy) and bring about better and more desirable results such as shortening of the required treatment period as well as shortening of the period for recovery, less damage to adjacent tissues, less discomfort to the subject being treated and others.

U.S. Pat. No. 6,702,808 to the same assignee discloses, for example, a device and method for treating skin employing both RF electrodes and optical energy, for example, to destroy hair follicles. In one example, the combined RF energy and light energy are both aimed at a target area (e.g., hair and hair follicle) having a diameter slightly over 100 microns (the diameter of a hair shaft). US Pat. Application Publication No 2011/0015549A1 to the same assignee also discloses an example of combined use of RF energy and light energy for treatment of diseased nails. In this case, treatment of diseased nails can benefit from the combined use of RF and light energies since the width of an average nail plate at its widest portion is close to the maximal efficient distance between electrodes which is about 1 cm.

As shown in the above examples, The concurrent use of RF energy and light energy for cosmetic treatment of skin, though being highly efficient and in some cases—desirable, may prove to be limited and beneficial only in treatments that lend themselves to small light energy spot sizes or more specifically to light energy spot sizes that can fit between two bipolar RF electrodes placed at a distance there between such that will result in effective conductive RF treatment. This is because, as stated above, the maximal distance between two RF electrodes to ensure effective conductive RF treatment is about 1 cm and a spot size having a greater diameter than 1 cm may bring about less effective skin treatment results due to the RF electrodes blocking the light energy from reaching skin directly under and in contact with the RF electrodes. This may also raise the need to correct the distribution of heat by continuously repositioning the RF electrodes.

Light spot size can be even further limited when employing a fractional (matrix) RF electrode for skin treatment as disclosed in U.S. Pat. No. 8,357,150 to the same assignee, especially since desired area to be treated can be quite large, for example 30×30 mm² or 40×40 mm².

As explained above, RF and light energies applied to skin act to increase the temperature of the treated skin area. In a large majority of case the temperature of the treated skin area is measured indirectly by delivering low power RF energy and determining the impedance of the treated area.

U.S. Pat. Nos. 6,702,808 and 8,357,150 also disclose a method of employing RF electrodes to measure a variation in skin impedance and extracting skin temperature from the received measurements. Since the treated area can be quite large, typically the skin temperature distribution in such large areas may not be uniform/homogenous. In such cases accurate skin temperature distribution measurement can become useful. However, when skin is treated with light energy additional to and concurrently with RF electrodes, such a configuration complicates the applicator structure, does not result in accurate skin temperature measurement and compromises skin temperature measurements in selected skin areas.

Commonly, applicators and especially electrodes for cosmetic treatment of skin are made of opaque materials such as metal coated plastic or metal limiting the operator's view of the segments of skin undergoing treatment.

### SUMMARY

Providing an applicator for cosmetic skin treatment that supports concurrent use of combined RF energy and light energy.

In one example, the applicator includes a transparent electrode assembly attached to the applicator and includes one or more transparent carrier and one or more transparent RF energy electrode elements attached to the carrier on a skin-facing surface thereof and supplied by a source of RF energy.

The transparent RF electrode can be positioned between the light source and skin to be treated and supports concurrent conductive RF and light energy treatment of segments of skin located directly under and in contact with the electrode elements of transparent electrode.

In one example, the transparent RF electrode can be electrically connected to a source of low power RF energy and measure skin temperature indirectly by determining impedance of a treated area.

In another example, the transparent RF electrode can have a “hot spot”-dispersing geometry.

In yet another example, the transparent RF electrode can have a “hot spot”-dispersing geometry in a form of an RF array of bi-polar pairs of RF energy applying elements.

In still another example, the transparent RF electrode can have a “hot spot”-dispersing geometry of two or more electrode components that spiral concentrically.

In another example, the transparent RF electrode can have a “hot spot”-dispersing geometry having a first and a second comb-like conductive RF conductive elements that have round-edged projections and wherein projections of the first electrode element are contactlessly disposed between projections of the second electrode element.

In still another example, the transparent RF electrode can be arranged in a form of a matrix (array) of transparent RF conductive elements disposed on non-conductive surface of a skin-contacting side of the carrier.

In another example, the transparent RF electrode can be arranged in a form of alternating strip conductive elements disposed on non-conductive surface of a skin-contacting side of the carrier.

### BRIEF DESCRIPTION OF THE DRAWINGS

In order to understand the apparatus and method and to see how it may be carried out in practice, examples will now be described, by way of non-limiting example only, with reference to the accompanying drawings, in which:

FIG. 1 is a partial diagram and cross-section view simplified illustration of a commonly used applicator for treatment of skin in accordance with an example;

FIG. 2 is a partial diagram cross-section view simplified illustration of an applicator for treatment of skin in accordance with another example;

FIG. 3 which is a cross-section view simplified illustration of a transparent electrode assembly for treatment of skin in accordance with yet another example;

FIGS. 4A, 4B and 4C, together referred to as FIG. 4, are plan-view simplified illustrations of a transparent conductive electrode configurations in accordance with an example;

FIG. 5 is a plan-view simplified illustration of another transparent RF electrode in accordance with still another example; and

FIG. 6 is a perspective-view simplified illustration of another transparent RF electrode in accordance with still another example.

### DETAILED DESCRIPTION

The concurrent use of RF energy and coherent (e.g., laser light energy) or incoherent light energy (e.g., Intense Pulsed Light or IPL) for cosmetic treatment of skin can be advantageous in some cases, such as, for example, in treatment of dark skin as well as provide a better safety profile and higher efficacy. Though being highly efficient in some cases, concurrent use of RF energy and light energy for cosmetic treatment of skin may prove to be limited and beneficial only in treatments that lend themselves to small light energy spot sizes or more specifically to light energy spot sizes that can fit between two bipolar RF electrodes placed at a distance there between such that will result in effective conductive RF treatment. This is because the maximal distance between two RF electrodes to ensure effective conductive RF treatment is about 1 cm and a spot size having a greater diameter than 1 cm may bring about non-uniform skin treatment results due to the RF electrodes blocking the light energy from reaching skin under the RF electrodes. This may also raise the need to continuously reposition the RF electrodes.

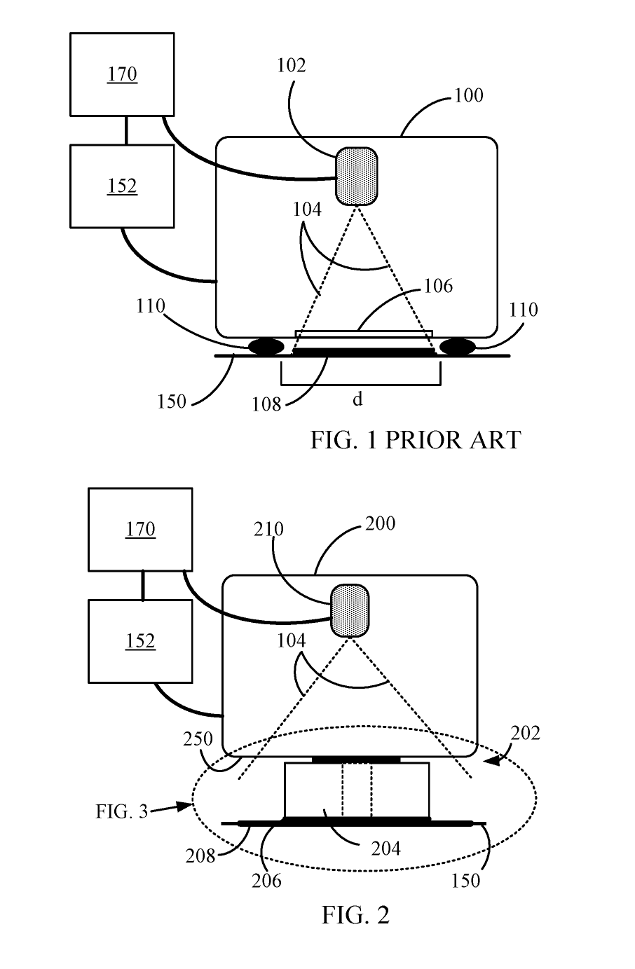

FIG. 1, which is a partial diagram and cross-section view simplified illustration of a commonly used applicator for treatment of skin in accordance with an example, depicts an applicator 100 designed for combined RF energy and light energy cosmetic treatment of skin. Applicator 100 can house a source of light energy 102 such as a laser, laser diode or IPL) applying a beam 104 of light the boundaries of which depicted by phantom lines projected through an aperture 106 in applicator 100 creating a spot 108 of light energy on skin 150. Applicator 100 can also house a pair of conductive RF electrodes 110 supplied by a source of RF energy 152. Sources 102 and 152 can both be controlled by a controller 170.

Commonly electrodes 110 are opaque to the light emitted by source of light energy 102 and when positioned between source 102 and a segment of skin 150, block the light emitted from source 102 casting a shadow on segments of skin 150 located directly under and in contact with electrodes 110, preventing light energy application to the skin beneath the electrodes bringing about lack of uniformity in light energy distribution over a treated portion of skin. Hence, it will be appreciated that the distance (d) between RF electrodes 110 can limit the size of light spot 108 to a diameter no larger than the maximal effective distance (d) between the two RF electrodes 110, which is about (d)=1 cm, to ensure effective conductive RF treatment.

Commonly, applicator 100 and electrodes 110 can be made of opaque materials also limiting the operator's view of the segment of skin 150 undergoing treatment.

As shown in FIG. 2, which is a partial diagram cross-section view simplified illustration of an applicator for treatment of skin in accordance with another example, an applicator 200 can support concurrent use of combined RF energy and light energy. Applicator 200 can have a transparent electrode assembly 202 attached to or deposited on a skin-contacting side 250 of applicator 200 and including a transparent carrier 204 and one or more transparent RF energy electrodes 206 supplied by a source of RF energy 152 and attached to or deposited on carrier 204 between a light source 210 and segment of skin 150 and in contact with segment of skin 150. Sources 102 and 152 can both be controlled by a controller 170.

Transparent carrier 302 can be shaped as a column, cube, trapezoid or in any other suitable geometric form and can be made of a non-conductive transparent material that supports transmission of light energy therethrough to the skin, such as borosilicate glass (e.g., Corning® Pyrex®), soda lime glass, sapphire, quartz or any other transparent non-conducting material and include one or more transparent RF energy electrodes 206 deposited on at least a portion of a non-conductive first surface 306 on a skin-contacting side of carrier 302.

Transparent carrier 302 can be selected so as to have good thermal conductivity to allow cooling of the skin surface. Materials such as sapphire and quartz could be used.

Transparent RF energy electrodes 206 can be made of an optically transparent and electrically conductive film (TCF) such as Indium Tin Oxide (ITO) and other transparent conductive oxides (TCOs), conductive polymers, metal grids, carbon nanotube (CNT), graphene, nanowire and ultra-thin metal films. Transparent RF energy electrodes 206 can be deposited on at least a portion of non-conductive first surface 306 on the skin-contacting side of carrier 302 by magnetron sputtering, metal organic chemical vapor deposition (MOCVD), metal organic molecular beam deposition (MOMBD), spray pyrolysis, ultrasonic nozzle sprayed graphene oxide and air sprayed Ag Nanowire and pulsed laser deposition (PLD) at a thickness that supports transmission of light energy from a light source such as light source 210 of FIG. 2 to a segment 370 of skin 150 directly under and in contact with Transparent RF energy electrodes 206 and sufficiently conductive so that to efficiently deliver RF energy while still maintaining sufficiently high transmittance, e.g. a transmittal range between 80% and 95% depending on the wavelength of the light energy to be used.

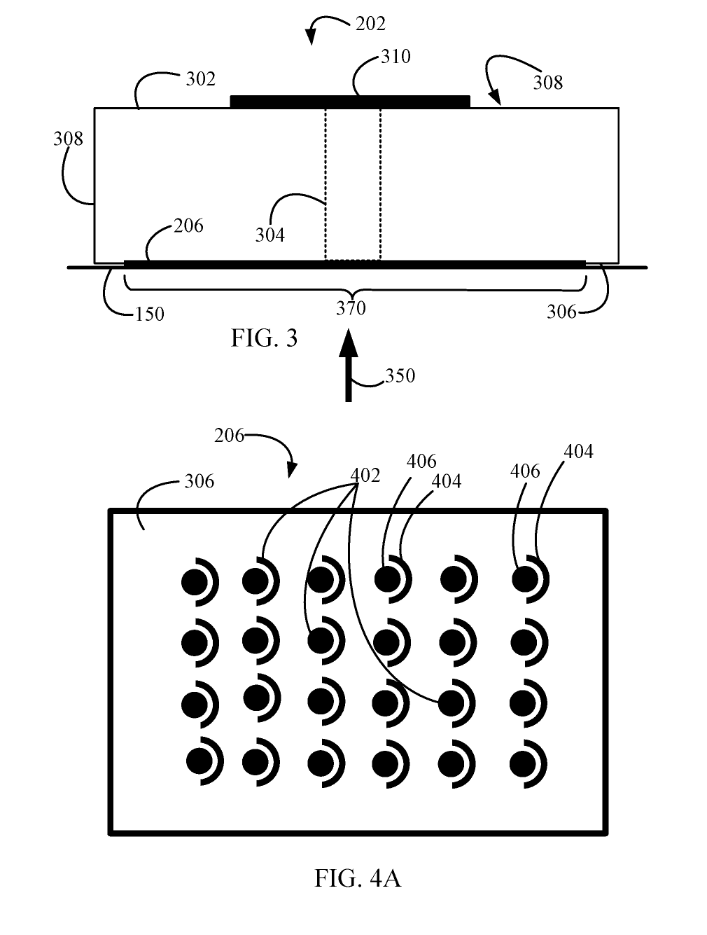

Employing a transparent electrode assembly 202 between light source 210 and segment of skin 150 removes any limitation mentioned above on a spot size 208 of light energy created by beam 104 on segment of skin 150. Transparent electrode assembly 202 and RF electrode 206 positioned between light source 210 and skin to be treated support treatment by light energy of segments of skin 150 located directly under and in contact with the electrodes of transparent electrode 206. This facilitates light energy to be transmitted through RF electrode assembly 202 and impinge on skin 150 including on segments of skin 370 (FIG. 3) located directly under and in contact with transparent electrode elements 502 so that to provide uniform heating of the skin. This allows an area of skin 150 being treated to be quite large for example, 30×30 mm² or 40×40 mm², supports uniform distribution of light energy over a segment of skin 150 being treated and provides good visualization of the segment of skin 150 undergoing treatment.

Referring now to FIG. 3, which is a cross-section view enlargement of the portion encircled in FIG. 2. As depicted in FIG. 3, transparent RF energy electrodes 206 can be electrically connected to a conductive surface 304 that can extend along a second non-conductive surface 308 on any side of, or through carrier 302 and be directly or indirectly electrically connected to one or more sources of RF energy 152 (FIG. 2) via one or more beryllium RF contacts 310 that can also support electrically connecting and/or structurally fixing carrier 302 to applicator 200 transparent electrode assembly 202.

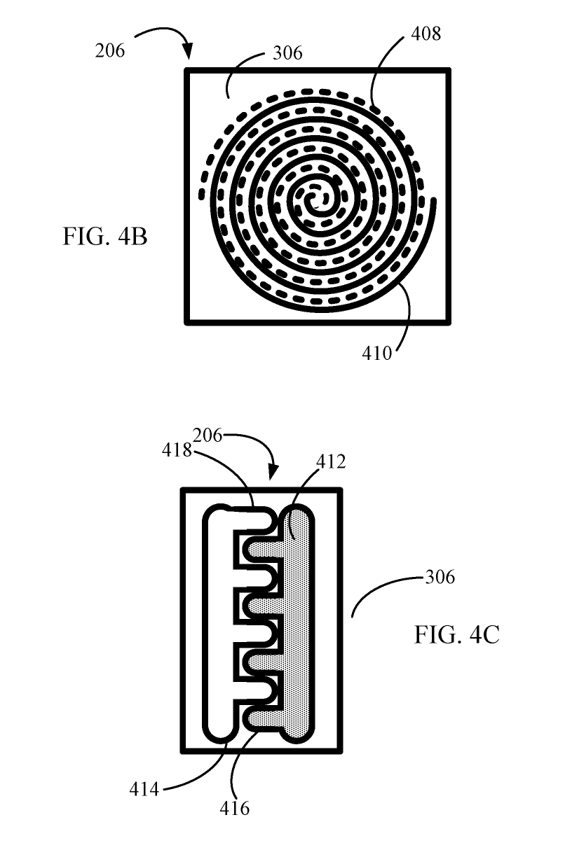

The transparent characteristic of electrode assembly 202, positioned between source of light 210 and skin to be treated supports light energy to be applied to segments 370 of skin 150 located directly under and in contact with the electrodes (FIG. 4) of transparent electrode assembly 202 and thus supports concurrent application of combined RF energy and light energy to uniformly heat segments of skin for cosmetic treatment and bring about better and more desirable results such as higher treatment efficiency by shortening of the required treatment period as well as shortening of the period for recovery, less damage to adjacent tissues, less discomfort to the subject being treated and others, as well as support good visualization of the treated skin segment.

As explained above, solutions to prevent the forming of “hot spots” commonly include various geometrical configurations of RF electrodes for example, configurations directed at eliminating as many sharp corners as possible of the electrode surface in contact with a segment of skin to be treated. However, many solutions can require relatively large electrode surface areas negating the option of concurrent use of both conductive RF and light energies in combination for the reasons explained above. However, employing a transparent electrode assembly such as the above disclosed transparent electrode assembly 202 removes this limitation and supports unhindered combined and concurrent use of both transparent non-“hot spot”-inducing conductive RF electrodes and light energy sources.

In some configurations elements of transparent RF energy electrodes 206 can have a “hot spot”-dispersing geometry. Transparent electrode 206 of electrode assembly 202 can have any type of “hot spot”-dispersing geometry and is not limited to any specific design or configuration. Electrode 206 depicted in FIGS. 4A, 4B and 4C, together referred to as FIG. 4, which are plan-view simplified illustrations of transparent conductive electrode configurations in accordance with an example, illustrate, but are not limited to, some examples of such types of non-“hot spot”-inducing transparent conductive RF electrode elements as viewed in a direction indicated by an arrow in FIG. 3 designated numeral 350.

One such example, shown in FIG. 4A, transparent electrode 206 can be configured in an array configuration of two or more RF energy applying elements 402, arranged in bi-polar pairs, each pair including one crescent-shaped element 404 and its counterpart being a disc-shaped element 406. RF energy applying elements 402 can be made of the same materials as Transparent RF energy electrodes 206 and be deposited via a mask or other known techniques on at least a portion of non-conductive first surface 306 by magnetron sputtering, metal organic chemical vapor deposition (MOCVD), metal organic molecular beam deposition (MOMBD), spray pyrolysis, ultrasonic nozzle sprayed graphene oxide and air sprayed Ag Nanowire and pulsed laser deposition (PLD).

The thickness of the electrode deposition can be at a thickness that will render RF energy applying elements 402 transparent and sufficiently conductive so that to efficiently deliver RF energy while still maintaining sufficiently high transmittance (i.e., over 90% transmittance). This due to the inverse relationship between transmittance and conductivity. E.g., the thicker the deposited electrode layer, the greater the conductivity of the layer but light transmittance is reduced proportionally.

RF energy applying elements 402 can be electrically connected as a group, directly or indirectly, to RF source of energy 152 and be controlled by controller 170. Alternatively and optionally, each individual pair of RF energy applying elements 402 can be electrically connected directly or indirectly to RF source of energy 152 and be controlled by controller 170. Each individual pair of one crescent-shaped elements 404 and its counterpart being a disc-shaped element 406 of RF energy applying elements 402 could be employed to measure local impedance between elements 404-406 and determine the local skin temperature. In FIG. 4, electrical connection arrangements have been removed for simplification of explanation.

In another example, shown in FIG. 4B, Transparent RF energy electrode 206 can be have a “hot spot”-dispersing geometry of two or more electrode components 408 and 410 that spiral concentrically. In FIG. 4B and for illustrative purposes only, a first electrode is depicted by a full-line spiral and a second electrode as a broken-line spiral. In this configuration, the “hot spot” can be dispersed in the area between the first and second electrode elements, throughout their entire length, reducing the level of heat generated at a single spot.

FIG. 4C depicts another example of RF electrode 206 having a “hot spot”-dispersing geometry in which a first and a second comb-like conductive RF conductive elements 412 and 414 respectively, have round-edged projections so that to eliminate sharp corners and edges and wherein projections 416 of first electrode element 412 are contactlessly disposed between projections 418 of second electrode element 414. Similarly to the configuration of FIG. 4B, in this example the “hot spot” can be dispersed in the area between the first and second “comb”-like electrode elements, throughout the entire circumference of the electrode aspects facing the other electrode, reducing the level of heat generated at a single spot.

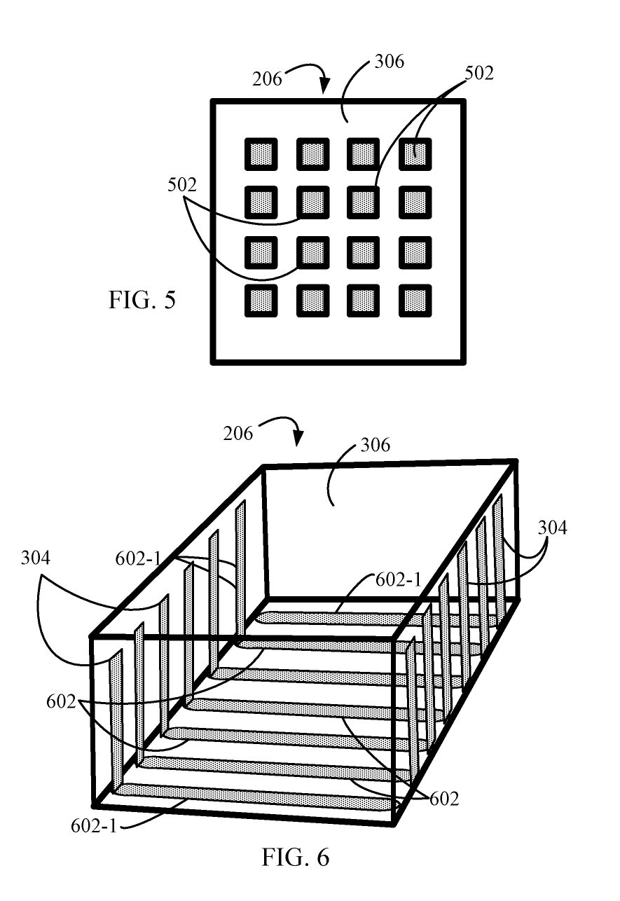

Reference is now made to FIG. 5, which is a plan-view simplified illustration of another transparent RF electrode in accordance with still another example. In a large majority of cases the temperature of the skin area being treated (i.e., heated) is measured indirectly by delivering low power RF energy and determining the impedance of the treated area.

FIG. 5 illustrates electrode 206 arranged in a form of a matrix (array) of transparent RF conductive elements 502 disposed on non-conductive first surface 306 of a skin-contacting side of carrier 302 (FIG. 3). Electrode elements 502 can be connected to RF energy source 152 and low power RF energy can be supplied to the electrode elements. Light energy could be transmitted through RF electrode assembly 202 and impinge on skin 150 including on segments of skin 370 (FIG. 3) located directly under and in contact with transparent electrode elements 502 so that to provide uniform heating of skin 150. In this configuration, fractionation of the RF heating distributes minute amounts of heating energy between electrode elements 502 and reducing the level of heat generated at a single spot. Additionally and optionally, the level of RF energy applied to the skin between each pair of electrode elements 502 can be adjusted to comply with local changes in skin parameters such as skin thickness, skin color, etc.

Additionally and optionally, pairs of RF conductive elements 502 can be used, concurrently with the application of light energy to homogenize the distribution of heat/skin temperature over a treated portion of skin heating areas in which measured skin treatment temperature is found to be lower than desired.

Additionally and optionally, skin impedance and hence temperature, can be measured between each pair of electrode elements 502 and/or between any arbitrary two electrode elements 502. The skin temperature data extracted from impedance measurements by a controller such as controller 170 of FIG. 2 can be displayed on a monitor and produce a skin temperature map of the portion of skin being treated.

Skin temperature maps can include a profile of the heating profile across the heated surface or in case where skin cooling is use, a cooling profile across the cooled surface.

Such method of skin temperature measurement is more reliable than the existing impedance measurement methods, since the gap between the electrode elements is small (millimeters or fractions of millimeters), and can be maintained as such since light energy can also be applied through transparent electrode assembly 202 to segments of skin 370 located directly under and in contact with electrode elements 502, applying uniform light energy to and thus uniformly heating the skin concurrently with and while skin temperature measurement are being taken by electrode elements 502. The skin temperature measurements could be continuous or with relatively high frequency to support real time skin temperature measurements.

Transparent RF energy applying elements 402 and electrode elements 408/410, 412/414 and 502 can be in electrical contact with transparent conductive surface 304 (FIG. 3).

FIG. 6, which is a perspective-view simplified illustration of another transparent RF electrode in accordance with still another example, shows strip conductive elements 602 having rounded edges to minimize creation of localized “hot spots” such as at sharp edges or points of the electrode element. Strip conductive elements 602 can be electrically connected via one or more conductive surfaces 304 individually or as a group, directly or indirectly, to RF source of energy 152 and be controlled by controller 170. Alternatively and optionally, each individual pair of strip conductive elements 602 can be electrically connected directly or indirectly to RF source of energy 152 and be controlled by controller 170. All or some of strip conductive elements 602 can be connected to two conductive surfaces 304, one on each end thereof so that to support switching the electrical polarity thereof and pairs of electrodes as desired. In FIG. 6, all strip conductive elements 602 but strip conductive elements 602-1 are connected to two conductive surfaces 304. In FIG. 6, electrical connection arrangements have been removed for simplification of explanation. Controller 170 is configured to switch RF source of energy 152 to a group or pairs of strip conductive elements 602.

It will be appreciated by persons skilled in the art that the present disclosure is not limited to what has been particularly shown and described hereinabove. Rather, the scope of the method and apparatus includes both combinations and sub-combinations of various features described hereinabove as well as modifications and variations thereof which would occur to a person skilled in the art upon reading the foregoing description and which are not in the prior art.

1. Applicator for cosmetic treatment of skin, comprising:
at least one source of light energy;
at least one transparent electrode assemblies positioned between the source of light energy and skin to be treated; and
wherein the transparent electrode assembly supports transmission of light energy from the light source to at least one segment of skin located directly under and in contact with the electrode assembly.

2. The applicator of claim 1, wherein a transparent electrode assembly supports concurrent application of combined RF energy and light energy to uniformly heat segments of skin for cosmetic treatment.

3. The applicator of claim 1, wherein a transparent electrode assembly comprises:
at least one transparent carrier made of a non-electrically conductive transparent material that supports transmission of light energy therethrough;
at least one transparent RF energy electrode elements deposited on at least a portion of a non-electrically conductive first surface on a skin-contacting side of the carrier; and
at least one RF contacts to provide electrical contact between the transparent RF energy electrode elements and a source of RF energy.

4. The applicator of claim 3, wherein the transparent carrier is made of at least one non-electrically conductive transparent material selected from a group of materials including borosilicate glass (e.g., Corning® Pyrex®, Eagle XG®), soda lime glass, sapphire and other similar transparent non-electrically conducting materials.

5. The applicator of claim 4, wherein a transparent electrode assembly comprises:
at least one transparent carrier made of a thermally conductive transparent material that supports transmission of light energy such as sapphire.

6. The applicator of claim 3, wherein the transparent RF energy electrode is made of at least one transparent and electrically conductive material selected from a group of materials including Indium Tin Oxide (ITO) and other transparent conductive oxides (TCOs), conductive polymers, metal grids, carbon nanotube (CNT), graphene, nanowire and ultra-thin metal films.

7. The applicator of claim 3, wherein the transparent RF energy electrode is deposited on the non-electrically conductive first surface on the skin-contacting side of the carrier by magnetron sputtering, metal organic chemical vapor deposition (MOCVD), metal organic molecular beam deposition (MOMBD), spray pyrolysis, ultrasonic nozzle sprayed graphene oxide and air sprayed Ag Nanowire and pulsed laser deposition (PLD).

8. The applicator of claim 3, wherein the transparent RF energy electrode is deposited on the non-electrically conductive first surface on the skin-contacting side of the carrier at a thickness that supports transmission of light energy from light source to a segment of skin directly under and in contact with the transparent RF energy electrode elements and be sufficiently conductive so that to efficiently deliver RF energy while still maintaining a transmittal range between 80% and 95%.

9. The applicator of claim 3, wherein the transparent RF energy electrode is in electrical contact with a conductive surface that extends along a second non-conductive surface on any side of, or through the transparent carrier and is directly or indirectly electrically connected to the one or more sources of RF energy via the one or more RF contacts.

10. The applicator of claim 3, wherein the RF contacts support electrically connecting and/or structurally fixing of the transparent carrier to the transparent electrode assembly.

11. (canceled)

12. The applicator of claim 3, wherein the electrode has a “hot spot”-dispersing geometry in a form of an RF array of bipolar pairs of RF energy applying elements.

13. (canceled)

14. The applicator of claim 3, wherein the electrode has a “hot spot”-dispersing geometry having a first and a second comb-like conductive RF conductive elements that have round-edged projections and wherein projections of first electrode element are contactlessly disposed between projections of second electrode element.

15. The applicator of claim 3, wherein the electrode has a “hot spot”-dispersing geometry having at least two strip RF conductive elements.

16-23. (canceled)

24. Applicator for cosmetic treatment of skin, comprising:
one or more sources of light energy;
one or more arrays of transparent RF conductive elements arranged in a matrix and positioned between the source of light energy and skin to be treated; and
wherein RF electrode elements are supplied with low power RF energy and indirectly measure skin temperature by measuring differences in skin impedance between each pair of RF electrode elements; and
wherein the transparent RF conducting elements supports transmission of light energy from the light source to at least one segment of skin located directly under and in contact with the electrode.

25. The applicator of claim 24, wherein transparent electrode supports concurrent application of light energy to uniformly heat segments of skin for cosmetic treatment and measurement of skin temperature.

26. The applicator of claim 24, wherein also comprising a controller that extracts skin temperature data from the impedance measurements.

27. The applicator of claim 24, wherein skin temperature measurements are continuous or with relatively high frequency to support real time skin temperature measurements.

28. The applicator of claim 24, wherein pairs of the matrix RF conductive elements are used, concurrently with application of light energy to homogenize distribution of heat/skin temperature over a treated portion of skin heating areas in which measured skin treatment temperature is found to be lower than desired.

29. Applicator for cosmetic treatment of skin, comprising:
one or more sources of light energy;
one or more transparent RF electrode elements positioned between the source of light energy and skin to be treated; and
wherein the transparent electrode supports transmission of light energy from the light source to one or more segments of skin located directly under and in contact with the electrode; and
wherein the applicator supports concurrent use of combined RF energy and light energy.
